(19) United States
(12) Patent Application Publication
(10) Pub. No.: US 2003/0155259 A1
(43) Pub. Date: Aug. 21, 2003

(54) Surgical suture needle counter

(76) Inventor: Tomoaki Koseki, Chiyoda-ku (JP)

Correspondence Address: Tomoaki Koseki, 17-2, Sotokanda 2-Chome, Chiyoda-ku, Tokyo, 101-0021, JP

(21) Appl. No.: 10/358,048

(22) Filed: Feb. 3, 2003

(30) Foreign Application Priority Data
Feb. 18, 2002 (JP) 2002-039437

(51) Int. Cl. A61B 17/06

(52) U.S. Cl. 206/380; 206/63.3; 206/363

(57) ABSTRACT

A surgical suture needle counter with the structure of having a needle fixing body made of thin plate formed of continuous projections and hollow parts in a waveform, wherein needles are inserted into the aperture of the projections and dropped into the hollow part to be fixed and held. Side rods protrude in both sides of the base of the hollow parts of the needle fixing body to clarify the precise position for the needle to be dropped when seen from above. The curved form of the hollow part of the needle fixing body is made to match the cross-section form of the needle to enable the inserted needle to fall into the aperture and fit closely onto the tray base. When the lid is open wide and turned to the base of the tray, the inside of the tray is inclined to facilitate easier visual confirmation of the fixed needles. The needle fixing body is mold processed of resin to be made a slightly longer than the tray ditch so that each projection part of the waveform adheres closely onto the tray, to increase the force to hold and fix the needles. Projections are provided to the base of the tray to grasp the needles, to obtain stronger force to hold and fix the needles. With the above points, this invention provides an affordable needle counter for surgical suture needles without using any magnetic material, having a strong force to hold and fix the needles, made to enable faster and easier visual confirmation of the number of needles after counting at one glance.

### BACKGROUND OF THE INVENTION

1. Field of the Invention

This invention relates to improvements of needle counters which count used needles for disposal to prevent any needle to be left in a patient's body, and also to appropriately dispose the used surgical suture needles as medical waste.

2. Description of the Prior Art

When a used suture needle is returned from a surgeon to a nurse, the needle is held in a various angles by a needle-holder. The nurse has to accurately and safely count the needles. Conventionally, there have been a various proposals for counting surgical needles such as placing needles on a magnet, pricking needles into a sponge, placing needles on specific partitioned space, or placing needles within an arch-shaped projection as proposed in claim 47 in Application Number 2001-306988.

### SUMMARY OF THIS INVENTION

When magnet is used, needles would obtain magnetic features, which is undesirable for surgery. Also, as the needle fixing force is not sufficient with the magnet method, needles tend to shift from the designated position. The sponge type is inconvenient in the fact that needles have to be re-held to prick into the sponge. Nurses' needle pricking accidents occur most when nurses have to re-hold the needles. In a case of a partition space needle counter type, it is difficult to confirm the number of needles at one glace after counting. Also in the partition type, if by accident the needle counter is turned over, there are risks of loosing needles and therefore the risk of infection. In the case of the needle counter proposed in claim 47 of Application Number 2001-306988, as the arch-shaped projection is directly projected from the base of the tray, the projection spring is hard and therefore it is difficult to insert a needle. Also in that case, as the distance between the projections are fixed, it is easy for a needle to drop off when the needle size changes. Also as adhesive pad is utilized in that invention to assist fixing, the manufacturing cost will be relatively high.

To solve the above problems, this invention consists of three parts being a tray, a lid, and a needle fixing body that is mounted inside the tray, wherein numbers for counting needles are inscribed at the side of the needle fixing body inside the tray, the needle fixing body is made of a thin plate of projections and hollow parts in a waveform, therefore when a used needle is inserted from an aperture between springy projections and dropped into the hollow part, the needle is fixed and held in that position.

Side rods are protruded from both side of the base of the hollow part of the needle fixing body, to clarify and mark the precise position to drop the needle in the hollow part when one looks at this counter from the top.

The curved form of the hollow part of the needle fixing body is in a structure to match the cross-section form of the needle to enable the inserted needle to drop into the base of the tray and fit closely onto the base part.

The tray and the lid are fixed with a hinge, and when the lid is open and turned over to the other side to have the upper side of the lid to contact the base side of the tray, the tray is made so that the inside of the tray can be seen clearly as the tray is inclined in relation to the horizontal level to facilitate better visual confirmation of the fixed needles.

The manufacturing method of this apparatus is such that a ditch is applied to the tray for fitting the needle fixing body, the needle fixing body is made of mold-processing from resin material to be a slightly longer than the ditch, so when the needle fixing body is adhered and fixed onto the tray, each projection and hollow parts of the wave shape adheres closely onto the tray, to increase the force of this apparatus to hold the needles.

Cut slits are made in each of the hollow part at the base of the needle fixing body, the cut slits fit inside the two pairs of projections one in the right and the other in the left which project to the center of the height of the needle fixing body from the inside base side of the tray, wherein the distance between each projection is equal to the diameter of the thinnest needle, so when a thinnest needle is dropped into the aperture of the projection part, the needle is fixed and held without any gap between the projections, and as the needle size becomes larger, the upper portion of the projection opens up, pressed between the upper portion of the inside of the hollow part of the needle fixing body, thence the needle is fixed and held.

### DESCRIPTION OF THE PREFERRED EMBODIMENTS

Hereafter, a description of the preferred embodiments of this invention is made in reference to drawings. FIG. 1 is a drawing of an embodiment of this invention according to claims 1, 2, 3, 4 and 5. Two rows of needle fixing bodies 1a and 1b adhered in parallel inside tray 2 are made of thin resin plates being in a waveform with continuous projections and hollow parts. The upper parts of needle fixing bodies 1a and 1b are projected, wherein a needle is inserted from an aperture between the projection part 5, the needle then drops into hollow part 6 and is fixed and held. The waveform with continuous projections and hollow parts shows spring effect alternately between each part, which enables easy inserting of the needle and strong fixing force.

Tray 2 is made of mold processing from white resin to facilitate easy visual confirmation of the needles. Lid 3 made of transparent resin is connected to the tray via a hinge, the lid folds to the back of tray 2 during usage, and is fixed. The inside of tray 2 is inclined towards the front side in relation to the horizontal level. As a nurse does not always handle a suture needle counter from right above or confirm the content of the counter from right above, it is effective to improve the visual confirmation condition with this inclination. As the inside of lid 3 becomes the base of this counter, it is necessary to apply rubber sponge or have projections and hollow parts made with mold processing and such for slip-proof. However, this apparatus needs to be visually confirmed from above once again before closing the lid after placing and fixing the needle, this slip-proof application should be conducted only to the surrounding of lid 3. Numbers for counting the needles are applied as embossed letters made of mold processing at the sides of needle fixing bodies 1a and 1b inside the tray.

Side rod 4 made of uniform mold processing is protruded from both sides of the base of the hollow parts of needle fixing bodies 1a and 1b. This side rod 4 will be the guide for precise position to drop in a needle. By setting the color of needle fixing bodies 1a and 1b in a dark color, it is easier to differentiate the parts of the needle fixing bodies from tray 2, thus there is no need for secondary printing process.

A suture needle is usually a curved needle made of columnar material, and the inside and the outside of the curve are made into a flat plane with press machine to facilitate the grasping of the needle with a needle holder. By setting the form of the hollow part of needle fixing bodies 1a and 1b so as to let stand the plane part of the suture needle, the inserted needle would automatically fall inside the counter, and fit closely onto the base of the tray. As the needle falls precisely into the counter, this secures safety of the usage of this counter as the needlepoint does not point above.

A ditch is applied with mold processing to the inside of tray 2 to mount the needle fixing bodies 1a and 1b. In relation to the ditch length, the needle fixing bodies 1a and 1b before fitting the bodies into the tray have a slightly longer length than the ditch, and each projection part within the waveform are slightly apart before fabrication. This is because it is impossible not to have any gaps when carving with mold processing. By mounting and installing the needle fixing bodies 1a and 1b onto the ditch on the tray, each projection part of the waveform are pressed and adhered closely, thus to increase its ability to hold a needle firmly.

FIG. 2 is an embodiment of a needle fixing body 1 according to claim 6. There are cut slits at the base of needle fixing bodies 1a and 1b, two pairs of projections 7 which projects to the center of the height of needle fixing bodies 1a and 1b are uniformly mold processed with tray 2, the cut slit is made to let the needles to fit into these slits from inside the tray. The distance between each projection in the vertical direction for this projection 7 is equal to the diameter of the thinnest needle, and in a case when a needle with the thinnest needle size is dropped into the aperture between projection 5, the needle will be fixed and held between the projections without any gaps. As the needle size becomes larger, the upper portion of the projection opens, the needle is pressed between the upper part of the inside of the hollow part 6 of needle fixing bodies 1a and 1b, thus the needle is more firmly fixed and held.

As in the above description, this invention enables the following effects.

As magnet is not used, the apparatus or needles would not obtain magnetic features. There is no need for a nurse to re-hold needles. After counting the needles, the apparatus can be sealed and closed, and the number of needles can be reconfirmed at a glance. There is less risk of loosing needles or of infection when turning over the needle counter. The waveform continuous projections and hollow parts cause springy effects alternately. By pressing a needle between projections on the tray, it is easy to drop in a needle even when the needle size changes, and the force to fix and hold the needles can be strong. The inside of the tray is inclined, enabling easier visual confirmation of the content. As side rods protrude to both sides at the base of the hollow part of the needle fixing body, the rods can be the precise guide for the position to drop in the needle. As the form of the hollow part of the needle fixing body matches the cross-section form of the suture needle, the needle automatically falls into the hollow part, fit closely to the base of the tray, and this would secure the safety. The ditch to which the needle fixing body is mounted inside the tray is made a slightly shorter than the needle fixing body itself, therefore when the needle fixing body is mounted inside the tray, each projection part of the waveform becomes pressed mutually and is firmly adhered, to increase the force to hold the needle.

### BRIEF DESCRIPTION OF THE DRAWINGS

FIG. 1 is an external drawing of a surgical suture needle counter.

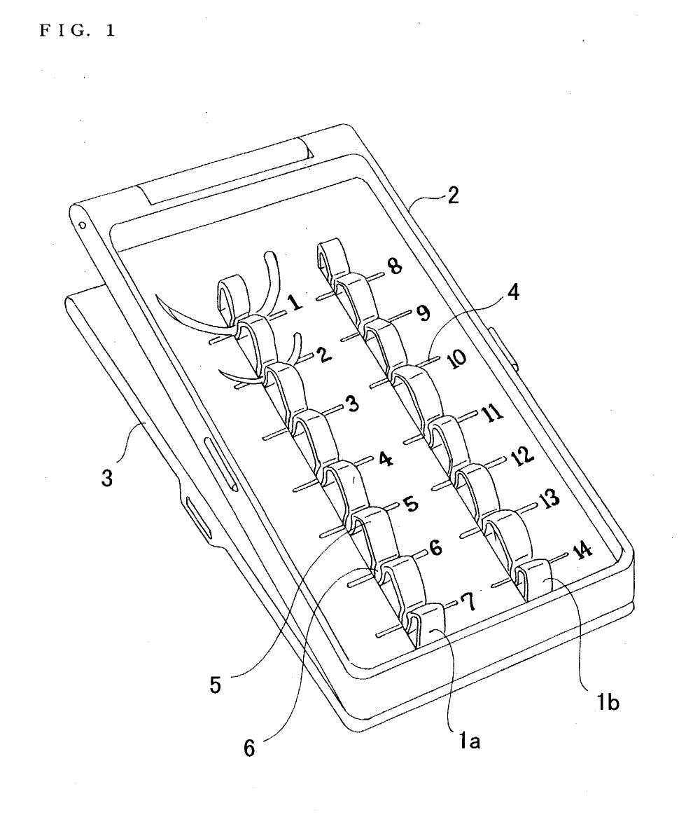

FIG. 2 is an enlarged drawing of a needle fixing body of this invention.

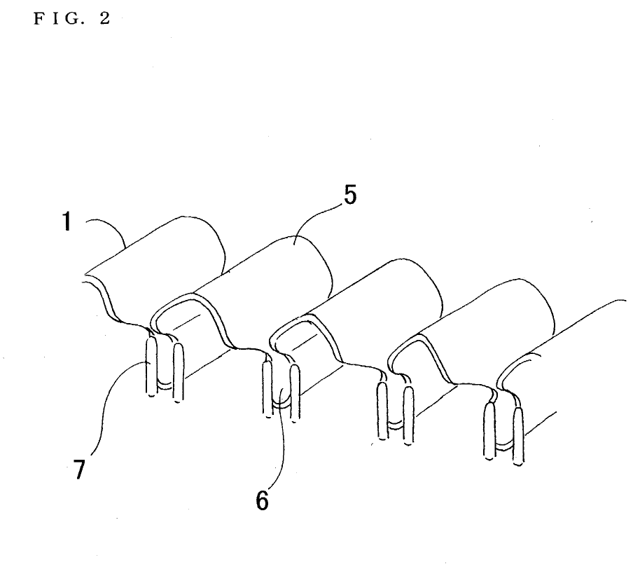

What is claimed is:

1. A surgical suture needle counter which is a container to count the number of used surgical suture needles and to dispose the used needles, which consists of a tray, a lid, and of needle fixing bodies mounted inside the tray, wherein the needle fixing body is made of a thin plate forming continuous waveform projections and hollow parts, needles are inserted into the aperture between the projections that have springy features, the needles are dropped into the hollow part and fixed there, numbers to count the needles are inscribed on the side of the needle fixing body inside the tray, the lid is closed after counting the needle for disposal.

2. A surgical suture needle counter according to claim 1, with the feature of side rods protruded on both sides at the base of the hollow parts of the needle fixing body, to clarify the precise position for the needle to be dropped looking at the needle counter from the top.

3. A surgical suture needle counter according to claims 1 and 2, wherein the curved form of the hollow part of the needle fixing body is made to match the cross-section form of the needle, to enable the inserted needle to drop into the needle fixing body and fit closely onto the base of the tray.

4. A surgical suture needle counter which is a container to count the number of used surgical suture needles and to dispose the used needles, which consists of a tray, a lid, and of needle fixing bodies mounted inside the tray, wherein the tray and the lid are fixed with a hinge, so when the lid is open and turned over to the other side to enable the upper part of the lid to contact the base of the tray, thus the inside of the tray is more visible as being inclined in relation to the horizon level, to increase the visibility of the content of the needle counter, and the needle fixing body is made of a thin plate forming continuous waveform projections and hollow parts, needles are inserted into the aperture between the projections that have springy features, the needles are dropped into the hollow part and fixed there, side rods protrude on both sides of the base of the hollow parts of the needle fixing body to clarify the precise position to drop in the needle, the curved form of the hollow part of the needle fixing body is made to match the cross-section form of the needle, to enable the inserted needle to drop into the needle fixing body and fit closely onto the base of the tray, numbers are inscribed on the side of the needle fixing body inside the tray to count the needles, the lid is closed and sealed after counting the needles for disposal.

5. A manufacturing method for a surgical suture needle counter according to claim 4, wherein a ditch is applied onto the tray to mount needle fixing bodies, a needle fixing body is made of mold processing of resin material made slightly longer than the tray ditch, and when the needle fixing body is mounted onto the tray ditch, each projection part of the waveform adheres closely to increase the force to hold and fix needles.

6. A surgical suture needle counter according to claims 4 and 5, wherein a cut slit is made at the base of the hollow part of the needle fixing body, the cut slit parts are placed within the two pairs of projections projected to the right and to the left sides which protrude up to about the center part of the height of the needle fixing body from inside the tray, the distance between each projection is equal to the diameter of the thinnest needle, and so when the needle dropped into the aperture of the projection is the thinnest size, the needle is firmly fixed without any gap between projections, and as the needle size becomes larger the upper part of the projection opens, and the needle is grasped between the upper parts of the inside of the hollow parts of the needle fixing body to firmly fix and hold the needles.
